(19) United States
(12) Patent Application Publication
(10) Pub. No.: US 2009/0299376 A1
(43) Pub. Date: Dec. 3, 2009

(54) Sensing Device for Medical Procedure

(76) Inventors: Jaime E. Martinez, Lincoln Park, NJ (US); Jose Villafane, Barranquilla (CO)

Correspondence Address: GIBBONS P.C., ONE GATEWAY CENTER, NEWARK, NJ, 07102, US

(21) Appl. No.: 12/127,234

(22) Filed: May 27, 2008

(51) Int. Cl. A61B 17/88

(52) U.S. Cl. 606/102; 606/53

(57) ABSTRACT

A sensing device for determining the location of an intramedullary pin emplaced within the bone of a patient. The sensing device is useable with a frame that initially holds the intramedullary pin external of the patient and the sensing device used to contact a planar portion of the intramedullary pin to verify the orientation of predrilled holes in the intramedullary pin. The same set up is thereafter used with the intramedullary pin emplaced within the bone of a patient. The sensing device verifies the same location of the predrilled holes by contacting the same planar portion. The sensing device is an electrical continuity device that completes an electrical circuit when the distal end of the sensing device contacts the planar portion of the intramedullary pin, thereby activating an alerting device to notify the physician that the contact has been established and the predrilled holes are accurately located.

### FIELD OF THE INVENTION

The present invention relates to a sensing device for use with a medical procedure and, more particularly, to a device to aid in the accurate location and orientation of an intramedullary pin.

### BACKGROUND

In the healing of certain fractures in bones of a patient, particularly the femur and tibia bones, one common procedure is the surgical emplacement of intramedullary pins within the bones. In such procedure, the intramedullary pin is normally retained in the desired position within the bone by means of securing screws that pass through the bone and pre-drilled holes in the intramedullary pin at both the distal and proximal ends of the intramedullary pin.

One of the difficulties, therefore, is to make sure that pin is in the exact desired location for receiving the screws since it is a blind procedure and the physician cannot actually see the intramedullary pin or the location of the pre-drilled holes in that pin.

One of the present methods of locating the pin position and the location of the holes therein involves the use of an image intensifier, however, that is a relatively costly device in that setting and hampers the surgeries.

Another currently used system involves an external frame that is set up prior to inserting the intramedullary pin onto the bone and the location of the holes in the distal end of the intramedullary pin is accomplished with a metal stick which fastens to and controls the correct position of the external frame system whenever that metal stick contacts the midline of the longitudinal axis of the intramedullary pin.

Again, however, since this is a blind procedure, it is difficult to evaluate and verify with total accuracy, the metal sound as the metal stick contacts the intramedullary pin that is indicative of the correct placement of the intramedullary pin contacting the midline of the longitudinal axis of the intramedullary pin.

The difficulty is, however, that with the metal stick is inserted, there must be a certainty that it is touching the intramedullary pin and further, where that contact takes place. In more the 50% of the cases, the extremity of the metal stick is wedged medially or laterally in relation to the intramedullary pin and thus allows a misalignment of the holes in the intramedullary pin when the screws are attempted to pass through those holes.

Since the bone's cortical area is solid, the contact between the metal stick and the solid cortical area could often be confused with contact with the intramedullary pin. Alternatively, the metal stick could be contacting the intramedullary pin in a lateral or offset location in relation to the midline of the intramedullary and therefore not at the midline itself. The problem is exacerbated by the fact that the intramedullary pin changes position when it is inserted into the medullary canal.

To a large degree, therefore, the effectiveness of locating the position and orientation of the distal end of the intramedullary pin is determined by the skill for the surgeon.

It would, therefore, be advantageous to have a relatively simple system that can be used to accurately locate the position and orientation of the intramedullary pin to verify that the holes in the distal end are properly positioned and aligned to receive the securing screws.

### SUMMARY OF THE INVENTION

Accordingly, the present invention relates to a sensing device and system for accurately determining the location and orientation of an intramedullary pin surgically emplaced within a bone of a patient.

The sensing device has a distal end and a proximal end and has a handle located at the proximal end. In the exemplary embodiment, the handle has an internal compartment within which there is an electronic circuit and an electrical source to power that electronic circuit. The electronic circuit has first and second electrical contacts such that when an electrical circuit is completed between both contacts, a signal is provided to activate an alerting device to alert a physician that the electrical circuit has been completed.

While the exemplary embodiment includes the presence of a handle, it can be seen that the sensing device of this invention could be manipulated by means of some automatic device, such as a robotic system, and thus not require presence of a handle.

As such, one contact is electrically connected to the intramedullary pin and the other is electrically connected to the distal end of the sensing device.

Thus, when the distal end of the sensing device contacts the intramedullary pin, the circuit is completed and the alerting device is activated.

As an alternative, the contact between the distal end of the sensing device and the intramedullary pin can be carried out by other sensing system, such as by inducing an electromagnetic field surrounding the intramedullary pin and/or the distal end of the sensing device and an electrical circuit provided to sense the change in that electromagnetic field when contact is made between the distal end of the sensing device and the intramedullary pin.

The device is used in a system that determines the orientation of the intramedullary pin, first external of the bone of the patient and then with the intramedullary pin emplaced within the bone of the patient. The location of the holes in the intramedullary pin is therefore established by the system external of the patient and then repeated with the intramedullary pin emplaced within the bone and, in each instance, the orientation of the holes in the intramedullary pin are established by a contact of the distal end of the sensing device with a planar section on the intramedullary pin.

The system therefore eliminates the subjective determination by a physician of the contact between a sensing device and the intramedullary pin by providing a positive alert to the physician when that contact has been established, thereby providing an accurate means of determining the location and orientation of the pre-drilled holes in the intramedullary pin.

These and other features and advantages of the present invention will become more readily apparent during the following detailed description taken in conjunction with the drawings herein.

### BRIEF DESCRIPTION OF THE DRAWINGS

FIG. 1 is a perspective view of an exemplary intramedullary pin for emplacement into the bone of a patient;

FIG. 2 is a perspective view of an exemplary frame retaining an intramedullary pin utilizing the present invention;

FIG. 3 is a perspective view illustrating the use of the present invention with an exemplary intramedullary pin emplaced within the bone of a patient;

FIG. 4 is a perspective view of a sensing device in accordance with one embodiment of the present invention;

FIG. 5 is an exploded view of the handle portion of the FIG. 4 embodiment; and

FIG. 6 is a schematic view of an exemplary electronic circuit useable with the present invention.

### DETAILED DESCRIPTION OF THE INVENTION

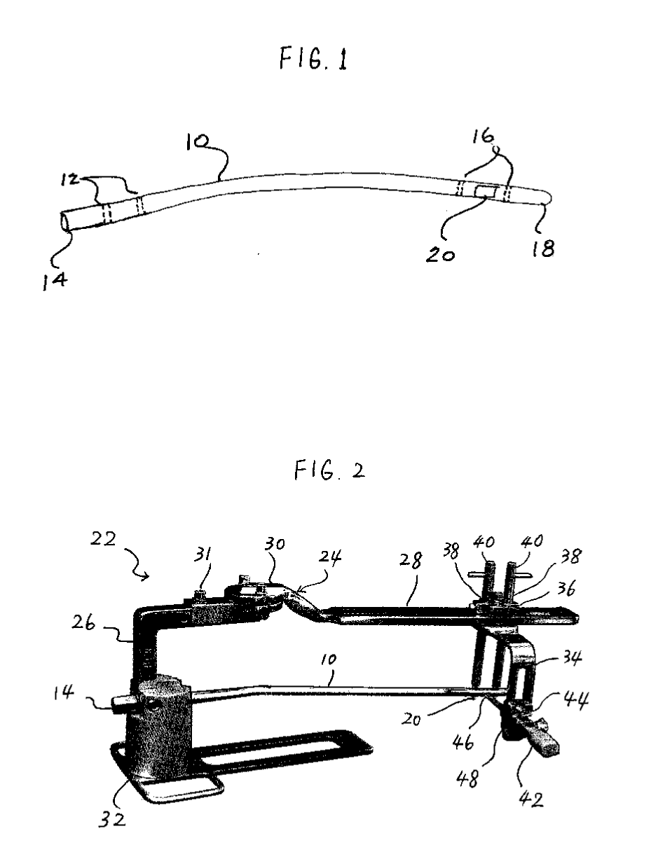

Referring now to FIG. 1, there is shown a perspective view of an exemplary intramedullary pin 10 that is to be emplaced within the bone of a patient. The intramedullary pin 10 is generally surgically emplaced within the tibia or femur bones to repair those bones and, in general, is comprised of, for example, a conductive metal, such as stainless steel, titanium and cobalt chrome.

In order to secure the intramedullary pin 10 within the bone of the patient, there are two holes 12 located at the proximal end 14 and two holes 16 located at the distal end 18. After the intramedullary pin 10 is surgically inserting into the bone, screws are passed through the holes 12, 16 to secure the intramedullary pin 10 within the bone. As such, since the intramedullary pin 10 is, at that time, located within the bone itself during the insertion of the screws, it is important that the physician be able to accurately locate the holes 12, 16 in order to properly align and insert the screws.

There is also a planar section 20 formed on the intramedullary pin 10 and that planar section 20 may preferably be in a plane that is parallel to a plane passing through the longitudinal axes of the holes 16.

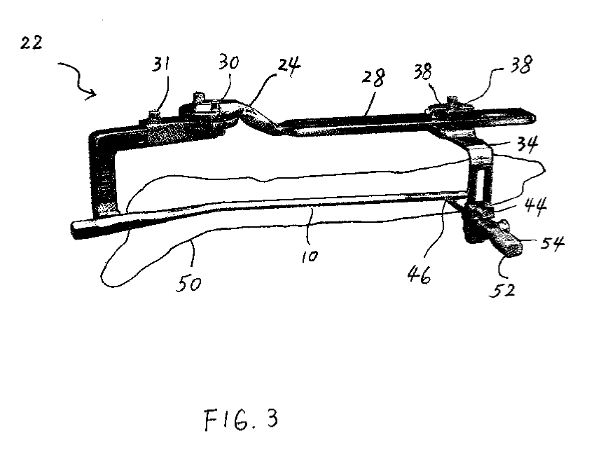

Turning next to FIG. 2, taken along with FIG. 1, there is shown a perspective view of a representative frame 22 that is used to determine the initial alignment of the intramedullary pin 10 so as to later accurately determine the location of the holes 16 when emplaced within the bone of the patient. The frame 22 includes generally horizontal strut 24 comprised of a proximal section 26 and a distal section 28 with the proximal and distal sections 26, 28 joined together by means of a movable joint 30 that allows side to side or pivoting movement therebetween.

The proximal section 26 is firmly affixed to the proximal end 14 of the intramedullary pin 10 whereas the intramedullary pin 10 itself is firmly affixed to a base 32. The distal section 28 is affixed to the proximal section 26 by means of the movable joint 30 and a bolt 31.

An L-shaped bracket 34 is disposed generally horizontally and slidably affixed to the distal section 28. The bracket 34 includes a boss 36 having two holes 38 that are spaced apart the same distance as the spacing of the holes 16 in the intramedullary pin 10. In the positioning of the frame 22, the distal section 28 is moved side to side to be in relative alignment with the intramedullary pin 10 and is then locked in that position at the movable joint 30.

The L-shaped bracket 34 can then be moved horizontally along the distal section 28 such that the holes 38 align precisely with the holes 16 in the intramedullary pin 10. To accomplish that goal, there are two alignment pins 40 that are passed through the holes 38 to enter into the holes 16 in the intramedullary pin 10. When that procedure has been completed, the location of the L-shaped bracket 34 is locked into position along the distal section 28.

At this point, the sensing device 42 of the present invention is inserted through a vertically movable guide 44 slidingly affixed to the L-shaped bracket 34 and the vertical movable guide 44 moved vertically until the distal end 46 of the sensing device 42 contacts the planar section 20 of the intramedullary pin 10. When that contact has been established, an alert is signaled to the user in a manner to be later explained.

In any event, when the alert is activated, the vertically movable guide 44 is locked in that position with respect to the L-shaped bracket 34 and a collar 48 tightened about the sensing device 42 marking its position with respect to the L-shaped bracket 34.

With that action, the exact location of the holes 16 is established with respect to the L-shaped bracket 34 and verified by the sensing device 42 and the frame is disassembled by separating the distal section 28 including the L-shaped bracket 34 affixed thereto by removal of the bolt 31 while leaving the proximal section 26 affixed to the intramedullary pin 10.

At this stage of the procedure, the intramedullary pin 10 can be surgically emplaced within the particular bone of the patient.

Turning then to FIG. 3, there is shown a perspective view illustrating the intramedullary pin 10 having been surgically emplaced within a bone 50 of a patient. The proximal section 26 of the horizontal strut 24 is still firmly affixed to the intramedullary pin 10 with the intramedullary pin 10 positioned within the bone 50.

At this point, the distal section 28 is reattached to the proximal section 26 be re securing the bolt 31 whereas the orientation of the distal section 28 is retained the same as was locked in by means of the movable joint 30 so that the location of the distal section 28, with the L-shaped bracket 34 still attached and in the same position with respect to the intramedullary pin 10 as described in FIG. 2.

A hole is then drilled in the bone 50 by inserting a drill through the vertically movable guide 44 in the same location as was used to insert the sensing device 42 so that the intramedullary pin 10 is accessible through hole drilled in the bone 50.

With the hole drilled in the bone, the sensing device 42 is again re-inserted through the movable guide 44 and maneuvered so as to make contact with the planar section 20 of the intramedullary pin 10. When that contact is established, there is a positive verification in the form of an alert to the physician that the sensing device 42 has made that contact and that, therefore, there is a positive indication that the holes 38 in the boss 36 are correctly and accurately positioned with respect to the holes 16 in the intramedullary pin 10.

A drill can now be used to pass through the holes 38 in the boss 36 and the drill will accurately align with the holes 16 in the intramedullary pin 10 so that holes can be drilled through bone 50 and passed through the holes 16. Once holes are drill, screws are inserted through the bone 50 and the holes 16 to secure the intramedullary pin 10 within the bone 50.

Accordingly, as can now be appreciated, an important component of the aforedescribed procedure is the sensing device 42 that is moved to a position contacting the planar section 20 of the intramedullary pin 10 to establish the correct and accurate position and alignment for drilling holes through the bone 50 that align with the holes 16 so that the screws can pass therethrough.

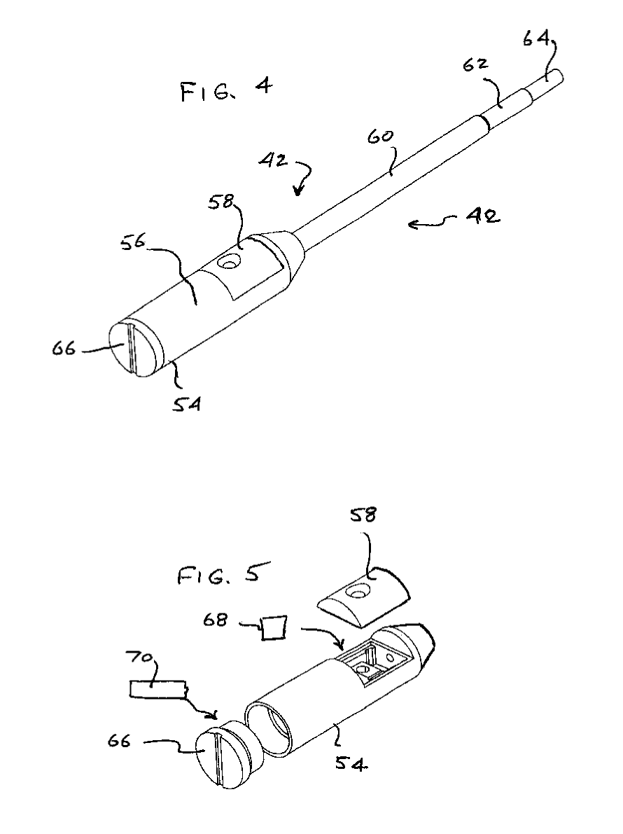

In FIG. 4, there is shown a perspective view of the sensing device 42 of the present invention. As can be seen, there is a distal end 46 and a proximal end 52. At the proximal end 52, there is a handle 54 for convenience in grasping and manipulating the sensing device 42 as previously described. In this exemplary embodiment, the handle 54 has an internal compartment 56 and has a cover 58 for access to the internal compartment 56. In the exemplary embodiment, the various components used to carry out the purpose of the present invention are located within the internal compartment 56. As can be seen, while the exemplary embodiment utilizes a handle, the present sensing device could be manipulated by means of some automatic device, such as a robotic system and thus eliminate the need for a handle.

Accordingly, there is an extension 60 extending distally from the handle 54 and can be affixed to the handle 54 by a threaded engagement. The extension 60 can electrically conductive and, in one exemplary embodiment, the extension 60 can serve as an electrical contact with the intramedullary pin 10 through the frame 22 as will be later described. An insulator 62 extends distally from the extension 60 and is comprised of an electrically insulating material. Finally, there is a conductive pin 64 that extends from the insulator 62 to the distal end 46 of the sensing device 42. The conductive pin 64 can extend proximally through the insulator 62 and the extension 60 to the internal compartment 56 of the handle 54. There is also a threaded cap 66 located at the proximal end of the handle 54 for access to the internal compartment 56.

Turning now to FIG. 5, there is an exploded view of the handle 54 having the threaded cap 66 and cover 58 removed, both providing the access to the internal compartment 56. As such, in the exemplary embodiment, an electronic circuit 68 is located within the internal compartment 56 along with an electrical source 70, such as batteries. Thus, in such embodiment, all of the components are located within the handle 54 that are needed to carry out the use of the sensing device 42.

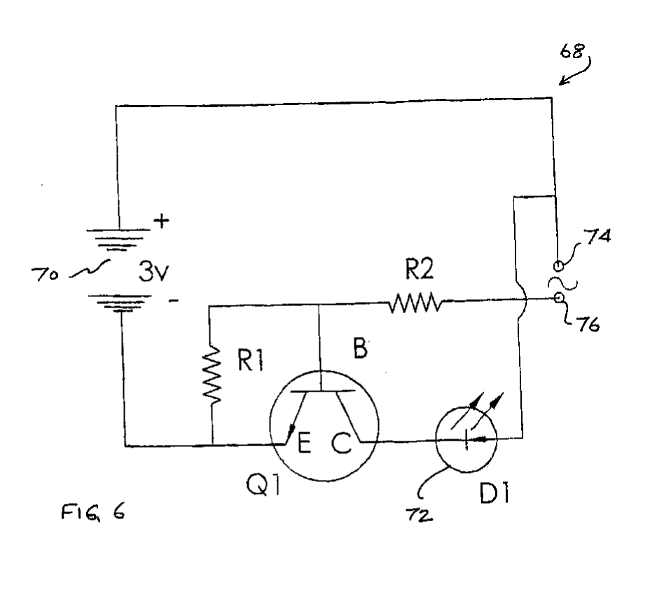

In FIG. 6, there is shown, a schematic view of an exemplary electronic circuit 68 useable with the present invention connected to an electrical source 70 and an alerting device 72. As can be seen, there are first and second contacts 74, 76 that, when electrically connected together, create an electrical circuit or signal to the alerting device 72. The alerting device 72 can be any device that has the ability of alerting the physician that an electrical circuit has been completed between the first and second contacts 74, 76 and may include a light signal, an audible signal or a vibrational signal.

As such, the electrical circuit 68 comprises a continuity checker such that when the first and second contacts 74, 76 complete a circuit, the alerting device 72 notifies the physician of that completed circuit. Thus, with the first contact 74, for example, electrically connected to the conductive intramedullary pin 10 and the second contact 76 electrically connected to the conductive pin 64 at the distal end 46 of the sensing device 42, any contact between the distal end 46 of the sensing device 42 and the intramedullary pin 10 will activate the alerting device 72 to notify the physician of that contact.

Returning to FIGS. 2 and 3, therefore, it can be seen that as the sensing device 42 is inserted through the vertically mounted guide 44, the alerting device 72 will be activated upon the distal end 46 of the sensing device 42 contacting the planar section 20 of the intramedullary pin 10 thereby assuring the physician that the holes 38 in the boss 36 are accurately aligned with the holes 16 in the distal end of the intramedullary pin 10.

As also can be seen in the exemplary embodiment, the electrical connection between the first contact 74 and the intramedullary pin 10 can easily be carried out by an electrical connection between that first contact 74 and the conductive extension 60 such that when that sensing device 42 is affixed to the vertically movable guides 44, the extension electrically contacts the frame 22, thereby establishing a electrically conductive path to the intramedullary pin 10. Alternatively, there may be a wire, not shown, that connects the first contact 74 to the frame 22 or any conductive portion of the frame or the intramedullary pin 10 to establish the electrical path.

As an alternative, while the exemplary embodiment senses the contact between the distal end 46 of the sensing device 42 by utilizing electrical continuity, it can be seen that other electrical systems could be used, for example there may be an electromagnetic field established surrounding the intramedullary pin 10 and/or the distal end 46 of sensing device 42 such the electrical circuit determines the contact between the distal end 46 of the sensing device 42 and the intramedullary pin 10 by sensing a change in that electromagnetic field.

Those skilled in the art will readily recognize numerous adaptations and modifications which can be made to the sensing device and its use of the present invention which will result in an improved device and system, including, for example, omission of the handle or locating the electronic circuit, alert indicator elsewhere in accordance with the present invention. In addition, it is possible to implement the invention by including a single contact for the sensory device for sensing an electrical signal induced in the intramedulary pin by means other than such electrical circuit yet all of which will fall within the scope and spirit of the present invention as defined in the following claims. Accordingly, the invention is to be limited only by the following claims and their equivalents.

What is claimed is:

1. A sensing device for use in emplacement of an intramedullary pin within a bone of a patient comprising:
an electronic circuit with a first contact located at distal end of the device; and
an alerting device electronically connected to the electronic circuit, wherein, in use, the electronic circuit activates the alerting device when the first contact touches an intramedullary pin.

2. The sensing device of claim 1 wherein the electronic circuit further comprises a second contact adapted to be being electrically connected to an intramedullary pin

3. The device of claim 1 further comprising a handle located at a proximal end of the device.

4. The sensing device of claim 3 wherein the electrical source is located within the handle.

5. The sensing device of claim 3 wherein the electronic circuit is located within the handle.

6. The sensing device of claim 3 wherein the device includes an extension section having an electrically conductive exterior surface.

7. The sensing device of claim 6 wherein the second contact is adapted to be electrically connected to the intramedullary pin through the extension section.

8. The sensing device of claim 6 including an insulated section intermediate the conductive extension section and the distal end.

9. The sensing device of claim 1 wherein the alerting device provides at least one of a light signal, a vibration or an audible signal.

10. A method for use in emplacement of an intramedullary pin within a bone of a patient comprising the steps of:
providing a sensing device and an electrical electronic circuit having a first contact disposed at a distal end of the device;
providing an electrical signal to said intramedullary pin;
producing the distal end of the device through the bone;
producing an alert when said sensing device contacts the intramedullary pin by sensing the electrical signal of said intramedullary pin.

11. The method of claim 10 wherein the step of producing an alert comprises producing an alert providing at least one of a light signal, a vibration and an audible signal.

12. The method of claim 9 wherein said electrical signal is provided to the intramedullary pin via a second contact of said electronic circuit.

13. A system for use with an intramedullary pin surgically emplaced within the bone of a patient and having holes in the distal end thereof; the system comprising:
a frame adapted to be located in close proximity to a human limb containing a bone with an intramedullary pin emplaced therein;
a sensing device affixed to the frame at a predetermined location, said sensing device having a proximal end and a distal end, the sensing device being movable between a withdrawn position and an inserted position wherein the distal end of the device contacts the intramedullary pin; and
an electronic circuit adapted to provide a signal upon the contact of the distal end of the sensing device with the intramedullary pin.

14. The system of claim 13 further comprising screws supported by the frame adapted to pass through the holes in the distal end of the intramedullary pin, said screws being aligned with the holes in the distal end of the intramedullary pin when said signal is present.

15. The system of claim 13 wherein the intramedullary pin has a planar section located in a plane generally parallel to a plane passing through the longitudinal axes of the holes.

16. The system of claim 13 wherein the sensing device contacts the planar section when in the inserted position.

17. The system of claim 13 wherein the electronic circuitry provides at least one of a light signal, a vibration or an audible signal.

18. The system of claim 13 wherein the sensing device has a handle located at the proximal end containing an electronic circuit and an electrical source.
